(19) United States
(12) Patent Application Publication
(10) Pub. No.: US 2022/0370005 A1
(43) Pub. Date: Nov. 24, 2022

(54) DERMATOSCOPE ATTACHMENT FOR MOBILE DEVICES

(71) Applicants: ALEXANDER M. WITKOWSKI, PORTLAND, OR (US); JOANNA N. LUDZIK, PORTLAND, OR (US)

(72) Inventors: ALEXANDER M. WITKOWSKI, PORTLAND, OR (US); JOANNA N. LUDZIK, PORTLAND, OR (US)

(21) Appl. No.: 17/853,889

(22) Filed: Jun. 29, 2022

Related U.S. Application Data
(63) Continuation of application No. 16/943,773, filed on Jul. 30, 2020.
(60) Provisional application No. 62/838,154, filed on Apr. 24, 2019.

(51) Int. Cl. A61B 5/00

(52) U.S. Cl. CPC A61B 5/441; A61B 5/0077; A61B 5/6898

(57) ABSTRACT

The dermatoscope attachment for mobile devices is a dermatoscope, which may be removably attached to a mobile device, such as a smartphone or the like, and includes an at least partially hollow housing having opposed front and rear faces with, respectively, a front window and a rear viewing port. An optical path is defined between the front window and the rear viewing port, and focusing optics are mounted within the at least partially hollow housing along the optical path. A light source is also mounted within the at least partially hollow housing for selectively projecting a light beam through the front window. A spring-biased clip is mounted to an upper end of the at least partially hollow housing for releasably holding a mobile device against the rear face of the at least partially hollow housing. Optionally, a rear window may be mounted in the rear viewing port.

### CROSS-REFERENCE TO RELATED APPLICATION

This application claims the benefit of U.S. Provisional Patent Application No. 62/838,154, filed on Apr. 24, 2019.

### BACKGROUND

### 1. Field

The disclosure of the present patent application relates to dermatoscopy, and particularly to a clip-on dermatoscope attachment for mobile devices, such as smartphones and the like.

### 2. Description of the Related Art

Dermatoscopy (also known as “dermoscopy” or “epiluminescence microscopy”) is the examination of skin features with a dermatoscope (also referred to as a “dermoscope”). A typical dermatoscope includes a magnifier (typically X10), a polarized light source and a transparent plate. By pressing the transparent plate against, or held near, the skin, the dermatoscope allows for inspection of skin lesions with a view which is unobstructed by skin surface reflections. Older dermatoscopes used non-polarized light and a liquid medium, but modern dermatoscopes dispense with the use of the liquid medium and instead make use of polarized light to cancel out skin surface reflections. When the images or video clips are digitally captured or processed, the instrument is sometimes also referred to as a “digital epiluminescence dermatoscope”.

Given the image processing, recording and transmission capabilities of modern portable devices, such as smartphones and the like, a number of different attachments for coupling dermatoscopes to various mobile devices are available. However, such attachments typically require the user to assemble and handle complicated and/or bulky components, such as specialized brackets, device covers and the like. Additionally, such attachments tend to be device-specific, i.e., for each different type of device, or for each different device model or brand, a separate attachment must be purchased. Thus, a dermatoscope attachment for mobile devices solving the aforementioned problems is desired.

### SUMMARY

The dermatoscope attachment for mobile devices is a dermatoscope, which may be removably attached to a mobile device, such as a smartphone or the like, and which may be universally used with a wide variety of differing types, models, styles and brands of mobile devices. Similar to a conventional dermatoscope, the dermatoscope attachment for mobile devices includes an at least partially hollow housing, having opposed front and rear faces. The front face has a front, optically transparent window mounted therein, and the rear face may also have a rear, optically transparent window mounted therein, or may simply have an empty viewing port defined therein adapted for alignment with the camera lens on the rear face of the mobile device. An optical path is defined between the front optically transparent window and the rear viewing port, or the rear optically transparent window (when present). Focusing optics, such as one more lenses, are mounted within the at least partially hollow housing along the optical path, and a light source is also mounted within the at least partially hollow housing for selectively projecting a light beam, such as a beam of polarized light, through the front, optically transparent window.

A spring-biased clip is mounted to an upper end of the at least partially hollow housing. The spring-biased clip is adapted for releasably holding a mobile device against the rear face of the at least partially hollow housing such that the rear viewing port (or optically transparent window) is positioned adjacent a camera lens of the mobile device. The spring-biased clip may include a pivoting arm, having opposed upper and lower ends, and a fixed arm. The fixed arm is secured to the upper end of the at least partially hollow housing, and the upper end of the pivoting arm is pivotally attached to, and spring-biased against, the fixed arm. The pivoting arm may be spaced apart from the rear face of the at least partially hollow housing for easily receiving the mobile device.

These and other features of the present disclosure will become readily apparent upon further review of the following specification and drawings.

### BRIEF DESCRIPTION OF THE DRAWINGS

FIG. 1 is a perspective view of a dermatoscope attachment for mobile devices as seen from the front of the device.

FIG. 2 is a side view of the dermatoscope attachment for mobile devices.

FIG. 3 is a perspective view of the dermatoscope attachment for mobile devices as seen from the rear of the device.

Similar reference characters denote corresponding features consistently throughout the attached drawings.

### DETAILED DESCRIPTION OF THE PREFERRED EMBODIMENTS

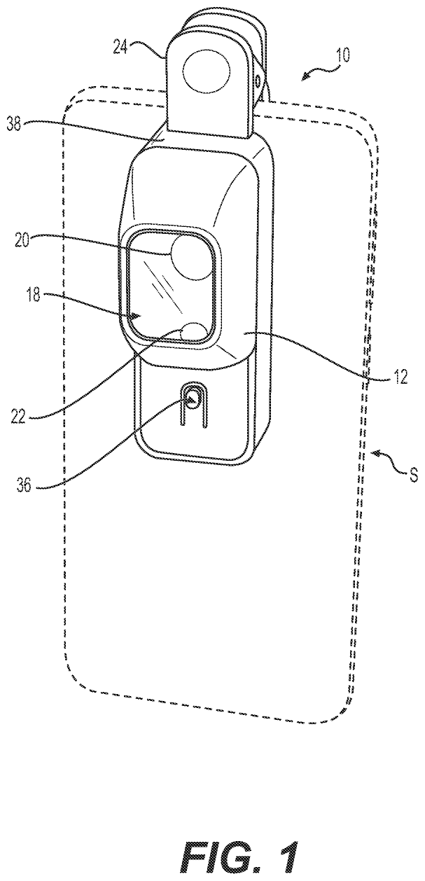

Referring now to FIG. 1, the dermatoscope attachment for mobile devices 10 is a dermatoscope, which may be removably attached to a mobile device, and which may be universally used with a wide variety of differing types, models, styles and brands of mobile devices. In FIG. 1, the dermatoscope attachment for mobile devices 10 is shown attached to an exemplary smartphone S. It should be understood that smartphone S is shown for exemplary purposes only, and that the dermatoscope attachment for mobile devices 10 may be used with any suitable type of mobile device, including, but not limited to, smartphones, tablets, laptop computers, personal digital assistants, digital cameras and the like.

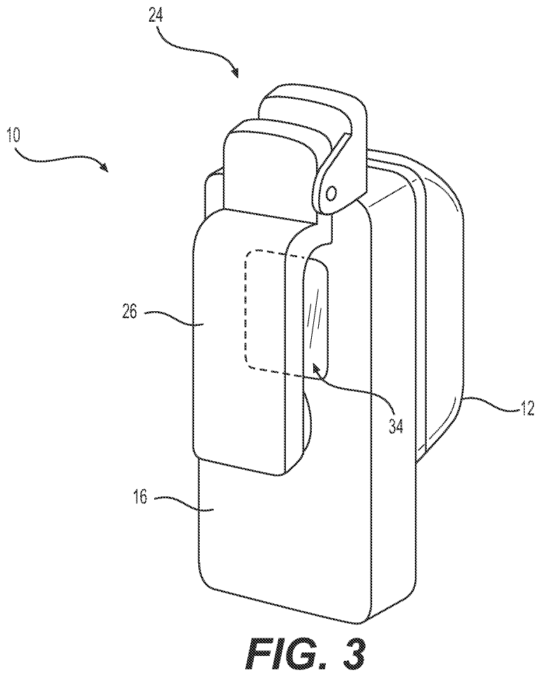

As shown in FIGS. 1-3, similar to a conventional dermatoscope, the dermatoscope attachment for mobile devices 10 includes an at least partially hollow housing 12, having opposed front and rear faces 14, 16, respectively. The front face 14 has a front, optically transparent window 18 mounted therein, and the rear face 16 may also have a rear, optically transparent window 34 mounted therein. Alternatively, although FIG. 3 shows the rear face 16 having a window 34 installed therein, optionally the window 34 may be omitted, leaving only a viewing port defined by the empty window opening in the rear face 16 for alignment of the front window 18 with the camera lens of the smartphone S or other mobile device. It should be understood that the overall configuration, size, shape and relative dimensions of housing 12 are shown for exemplary purposes only. Similarly, the size, shape, relative dimensions and positioning of the optically transparent windows 18, 34 are also shown for exemplary purposes only. Additionally, it should be understood that the housing 12 may be manufactured from any suitable type of durable material, such as a durable plastic or the like. Similarly, it should be understood that the optically transparent front window 18, and rear window 34 (when present), may be manufactured from any suitable type of durable and optically transparent material, such as clear, transparent plastic, glass or the like.

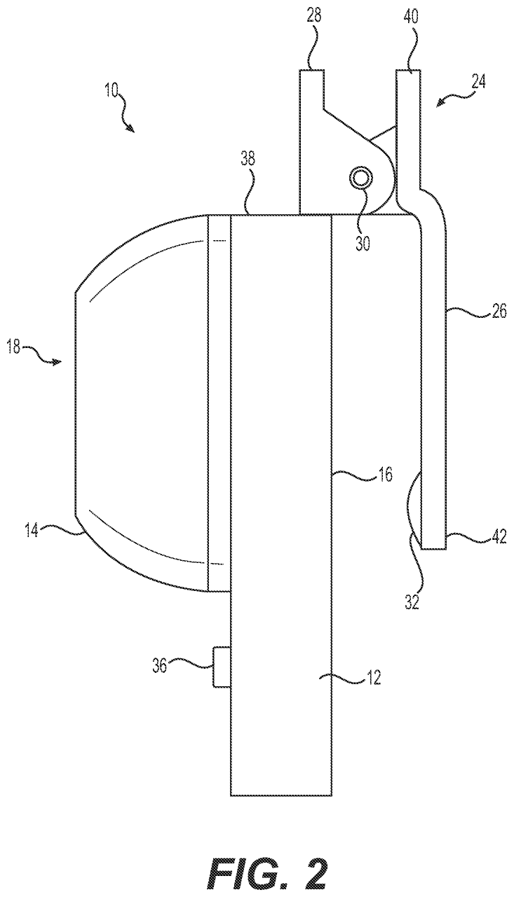

An optical path is defined between the front optically transparent window 18 and the open rear viewing port (or rear optically transparent window 34, when present). Focusing optics 20, such as one more lenses, are mounted within the at least partially hollow housing 12 along the optical path, and at least one light source 22 is also mounted within the at least partially hollow housing 12 for selectively projecting a light beam, such as a beam of polarized light, through the front optically transparent window 18. It should be understood that any suitable type of focusing optics may be used, depending upon the desired magnification factor for the dermatoscope attachment 10. It should be further understood that any suitable type of light source may be used, such as one or more light emitting diodes or the like. The light source 22 may be selectively actuated by button 36 or any other suitable type of switch, actuator or controller. It should be understood that the positioning, configuration, size and shape of button 36 are shown in FIGS. 1 and 2 for exemplary purposes only.

A spring-biased clip 24 is mounted to an upper end 38 of the at least partially hollow housing 12. The spring-biased clip 24 is adapted for releasably holding a mobile device, such as exemplary smartphone S, against the rear face 16 of the at least partially hollow housing 12, such that the rear viewing port (or optically transparent window 34, when present) is positioned adjacent a camera lens of the mobile device. As best seen in FIG. 2, the spring-biased clip 24 may include a pivoting arm 26 having opposed upper and lower ends 40, 42, respectively, and a fixed arm 28. The fixed arm 28 is secured to the upper end 38 of the at least partially hollow housing 12, and the upper end 40 of the pivoting arm 26 is pivotally attached to, and spring-biased against, the fixed arm 28. In FIG. 2, upper end 40 is shown as being spring-biased against, and pivotally attached to, the fixed arm 28 by a conventional spring-biased pivot 30 (e.g., a pivot pin or axle with a coaxially disposed torsion spring mounted thereon), although it should be understood that spring-biased clip 24 may be any suitable type of spring-biased clip. The pivoting arm 26 may be spaced apart from the rear face 16 of the at least partially hollow housing 12 for easily receiving the mobile device, and the lower end 42 of pivoting arm 26 may have a raised gripping portion 32, which may be formed from a soft and non-scratching material, such as foam rubber or the like, for effectively gripping the mobile device without causing damage thereto.

The attachment 10 may have dust covers releasably disposed over the front window 18 and/or the rear viewing port (or transparent window 34, when present). The housing 12 may have one or more batteries mounted therein for powering the light source 22. Additionally, in addition to raised gripping portion 32, it should be understood that the inner face of pivoting arm 26 and/or the rear face 16 of housing 12 may have padding or the like attached thereto in order to prevent damage to the smartphone S. It should be understood that any suitable type of padding or other protective material may be used, and it should be further understood that this padding or other protective material may be secured to the inner face of pivoting arm 26 and/or the rear face 16 of housing 12 using any suitable method.

It is to be understood that the dermatoscope attachment for mobile devices is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A dermatoscope attachment for mobile devices, comprising:
an at least partially hollow housing having opposed front and rear faces, the front face having a front window and the rear face having a rear window opening defining a rear viewing port, the front window and the rear viewing port defining an optical path therebetween;
focusing optics mounted within the at least partially hollow housing along the optical path;
a light source mounted within the at least partially hollow housing for selectively projecting a light beam through the front window; and
a spring-biased clip mounted to an upper end of the at least partially hollow housing, the spring-biased clip being adapted for resiliently retaining a mobile device against the rear face of the at least partially hollow housing such that the rear viewing port is positioned adjacent a camera lens of the mobile device.

2. The dermatoscope attachment for mobile devices as recited in claim 1, wherein the spring-biased clip comprises:
a pivoting arm having opposed upper and lower ends; and
a fixed arm secured to the upper end of the at least partially hollow housing, the upper end of the pivoting arm being pivotally attached to, and spring-biased against, the fixed arm.

3. The dermatoscope attachment for mobile devices as recited in claim 2, wherein the pivoting arm of the spring-biased clip has a raised gripping portion.

4. The dermatoscope attachment for mobile devices as recited in claim 2, wherein the pivoting arm is spaced apart from the rear face of the at least partially hollow housing.

5. The dermatoscope attachment for mobile devices as recited in claim 1, further comprising a rear window mounted in the rear viewing port.

6. The dermatoscope attachment for mobile devices as recited in claim 1, further comprising an actuator mounted on the front face of the at least partially hollow housing for selectively actuating the light source.

7. The dermatoscope attachment for mobile devices as recited in claim 1, wherein the light source comprises a polarized light source.
